(19) United States
(12) Patent Application Publication
(10) Pub. No.: US 2013/0271727 A1
(43) Pub. Date: Oct. 17, 2013

(54) OPHTHALMIC APPARATUS

(71) Applicant: CANON KABUSHIKI KAISHA, Tokyo (JP)

(72) Inventor: Shintaro Akiba, Isehara-shi (JP)

(73) Assignee: CANON KABUSHIKI KAISHA, Tokyo (JP)

(21) Appl. No.: 13/855,914

(22) Filed: Apr. 3, 2013

(30) Foreign Application Priority Data
Apr. 16, 2012 (JP) 2012-093398

(51) Int. Cl. A61B 3/00; A61B 3/18; A61B 3/117

(52) U.S. Cl. CPC A61B 3/0083; A61B 3/117; A61B 3/18
USPC 351/205; 351/245

(57) ABSTRACT

An ophthalmic apparatus includes an apparatus fixing unit, an optometric unit configured to move relative to the apparatus fixing unit, and a face support unit configured to fix an eye to be examined as an inspection target of the optometric unit. The ophthalmic apparatus includes: a face support moving unit configured to move the face support unit relative to the apparatus fixing unit; and an optometric unit moving unit configured to move the optometric unit relative to the apparatus fixing unit.

### BACKGROUND OF THE INVENTION

1. Field of the Invention

The present invention relates to an ophthalmic apparatus.

2. Description of the Related Art

As an ophthalmic apparatus which inspects a plurality of eye characteristics of an eye to be examined, there is known an apparatus which includes an eye pressure measurement unit which noncontactly measures an eye pressure and an ocular refractive power measurement unit which measures an ocular refractive power, and performs measurement by switching the units (Japanese Patent Laid-Open Nos. 2007-144128 and 2010-148589).

When measuring an eye pressure, in order to perform accurate measurement, it is necessary to make the eye to be examined firmly open. For this reason, an examiner often executes measurement while extending his/her own arm to make an eyelid of an object to be examined open, that is, so-called eyelid retraction. In the arrangements disclosed in Japanese Patent Laid-Open Nos. 2007-144128 and 2010-148589, however, the examiner faces the object through the measurement head portion, and hence has difficulty in seeing an eye to be examined and is distant from the eyelids of the objet. For this reason, for example, a female examiner who has a small body and short arms needs to perform operation in a difficult position, that is, perform measurement while making the eyelid open by fully extending her arm in a slightly tilted body.

The present invention has been made in consideration of the above problem and provides an ophthalmic apparatus which can improve the operability of eyelid retraction by an examiner and increase the degree of freedom of the installation layout of the apparatus.

### SUMMARY OF THE INVENTION

According to one aspect of the present invention, there is provided an ophthalmic apparatus including an apparatus fixing unit, an optometric unit configured to move relative to the apparatus fixing unit, and a face support unit configured to fix an eye to be examined as an inspection target of the optometric unit, the apparatus comprising: a face support moving unit configured to move the face support unit relative to the apparatus fixing unit; and an optometric unit moving unit configured to move the optometric unit relative to the apparatus fixing unit.

The present invention can improve the operability of eyelid retraction of an object to be examined and increase the degree of freedom of the installation layout of the apparatus.

Further features of the present invention will become apparent from the following description of exemplary embodiments (with reference to the attached drawings).

### BRIEF DESCRIPTION OF THE DRAWINGS

FIG. 1 is a view showing the schematic arrangement of an ophthalmic apparatus according to an embodiment;

FIG. 2 is a view showing the arrangement of the optical system of the optometric unit of the ophthalmic apparatus according to the embodiment;

FIGS. 3A and 3B are perspective views each showing an alignment prism stop of the ophthalmic apparatus according to the embodiment;

FIGS. 4A to 4C are plan views of the optometric unit of the ophthalmic apparatus according to the embodiment;

FIGS. 5A to 5C are plan views for explaining the moving mechanism of the face support unit of the ophthalmic apparatus according to the embodiment;

FIG. 6 is a block diagram showing the arrangement of the control system of the ophthalmic apparatus according to the embodiment;

FIGS. 7A and 7B are views for explaining the anterior ocular segment images captured by the ophthalmic apparatus according to the embodiment;

FIG. 8 is a flowchart for explaining the operation of the ophthalmic apparatus according to the embodiment at the time of optometry;

FIGS. 9A to 9C are views for explaining the disposition of the face support unit for optometry and optometric unit of the ophthalmic apparatus according to the embodiment;

FIGS. 10A to 10D are views for explaining the operation of the optometric unit of the ophthalmic apparatus according to the embodiment at the time of optometry; and

FIGS. 11A to 11D are views for explaining the operation of the optometric unit of an ophthalmic apparatus according to the second embodiment at the time of optometry.

### DESCRIPTION OF THE EMBODIMENTS

### First Embodiment

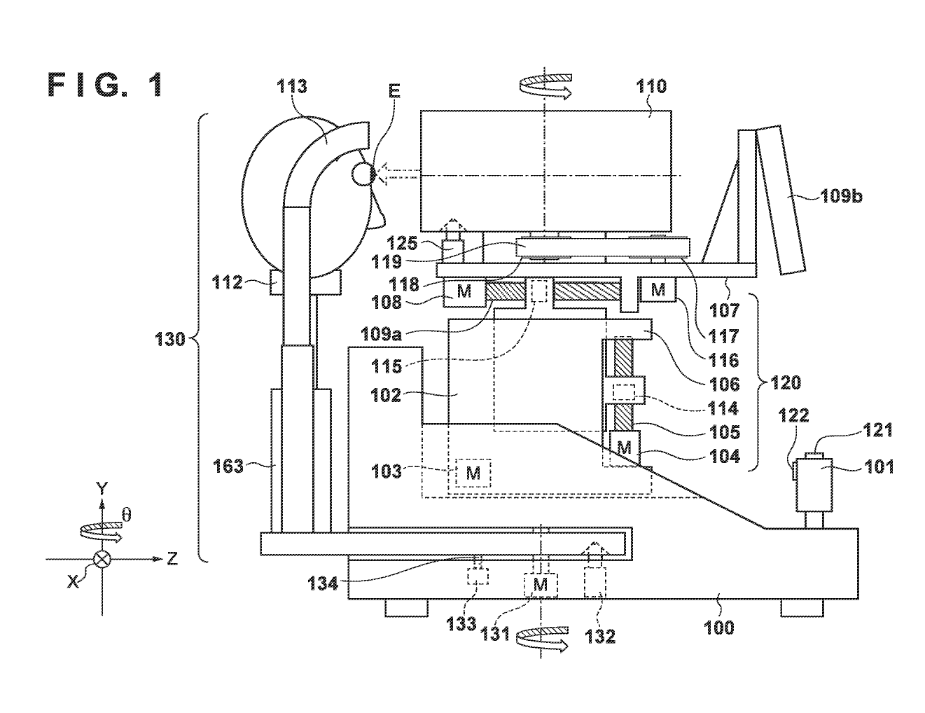

An ophthalmic apparatus according to an embodiment of the present invention will be described in detail with reference to the accompanying drawings. FIG. 1 is a view showing the schematic arrangement of the ophthalmic apparatus according to the embodiment. The ophthalmic apparatus includes a base 100 (apparatus fixing portion), a face support unit 130 for supporting the face of an object, and a driving unit 120 provided on the base 100. The ophthalmic apparatus also includes a joystick 101 as an operation member, a display unit 109b, and an optometric unit 110 (measurement unit) attached to the driving unit 120. The driving unit 120 includes driving mechanisms corresponding to the respective axes of the optometric unit 110 to move it in the X, Y, Z, and Θ directions.

(Movement in X-Axis Direction)

A frame 102 can move in the horizontal direction (to be referred to as the X-axis direction hereinafter) relative to the base 100. A driving mechanism in the X-axis direction includes an X-axis driving motor 103 fixed on the base 100, a lead screw (not shown) coupled to a motor output shaft, and a nut (not shown) which is fixed to the frame 102 and can move on the lead screw in the X-axis direction. As the X-axis driving motor 103 rotates, the frame 102 moves in the X-axis direction through the lead screw and the nut.

(Movement in Y-Axis Direction)

A frame 106 can move in the vertical direction (to be referred to as the Y-axis direction hereinafter) relative to the frame 102. A driving mechanism in the Y-axis direction includes a Y-axis driving motor 104 fixed on the frame 102, a lead screw 105 coupled to a motor output shaft, and a nut 114 which is fixed to the frame 106 and can move on the lead screw in the Y-axis direction. As the Y-axis driving motor 104 rotates, the frame 106 moves in the Y-axis direction through the lead screw and the nut.

(Movement in Z-Axis Direction)

A frame 107 can move in the back-and-forth direction (to be referred to as the Z-axis direction hereinafter) relative to the frame 106. A driving mechanism in the Z-axis direction includes a Z-axis driving motor 108 fixed on the frame 107, a lead screw 109a coupled to a motor output shaft, and a nut 115 which is fixed to the frame 106 and can move on the lead screw in the Z-axis direction. As the Z-axis driving motor 108 rotates, the frame 107 moves in the Z-axis direction through the lead screw and the nut.

(Rotation in Θ-Axis Direction)

The optometric unit 110 can move in the rotational direction (to be referred to as the Θ-axis direction hereinafter) relative to the frame 107. A driving mechanism (optometric unit moving unit) in the Θ-axis direction includes a Θ-axis driving motor 116 fixed on the frame 107 and a pulley 117 coupled to a motor output shaft. The driving mechanism in the Θ-axis direction includes a pulley 118 coupled to the optometric unit 110 and a belt 119 coupled to the pulley 117 and the pulley 118. As the Θ-axis driving motor 116 rotates, the optometric unit 110 rotationally moves around the rotation axis (Θ-axis direction) relative to the base 100 through the pulley 117, the belt 119, and the pulley 118.

(Positioning Stopper)

A stopper 125 (positioning member) for positioning the optometric unit is fixed on the frame 107. The stopper 125 has a wedge-shaped distal end. The stopper 125 is driven in the vertical direction to be inserted into a positioning groove portion provided in the lower portion of the optometric unit 110. The Θ-axis driving motor 116 is driven to rotationally move the optometric unit 110 in the Θ-axis direction. The stopper 125 is then inserted into the groove portion to position and fix the optometric unit 110 at a predetermined position.

(LCD Monitor)

The examiner-side end portion of the frame 107 is provided with an LCD monitor as the display unit 109b for observing an eye E to be examined as an inspection target of the optometric unit 110.

(Face Support Unit)

When performing optometry, the examiner can fix the position of the eye to be examined by letting the object rest his/her chin on a chin rest 112 and pressing his/her forehead against the forehead rest portion of a face support frame 113. The face support unit 130 is provided so as to be movable relative to the base 100. As a face support driving motor 131 (face support moving unit) fixed on the base 100 rotates, the face support unit 130 moves around the rotation axis (Θ-axis direction) relative to the base 100. After rotationally moving in the Θ-axis direction, the face support unit 130 is positioned/fixed at a predetermined position upon insertion of a positioning stopper 132 fixed on the base 100. The rotation axis for rotational movement of the driving mechanism in the Θ-axis direction (optometric unit moving unit) coincides with the rotation axis for rotational movement of the face support driving motor 131 (face support moving unit). A position detection sensor 133 (for example, a microswitch) fixed on the base 100 can detect the position of the face support unit 130 after movement. It is possible to move the position of the chin rest 112 by driving a chin rest driving motor 163. It is possible to raise or lower the chin rest 112 so as to adjust its position by driving the chin rest driving motor 163.

(Joystick)

The base 100 is provided with the joystick 101 as an operation member for positioning the optometric unit 110 relative to the eye E as an inspection target and an optometry switching button 122. The examiner instructs the driving direction, driving amount, and driving speed of the driving unit 120 by tilting/operating the joystick 101. Upon positioning (aligning) the optometric unit 110 relative to an eye to be examined as an inspection target, the examiner executes measurement by pressing a measurement start button 121 provided on the joystick 101.

(Optical System)

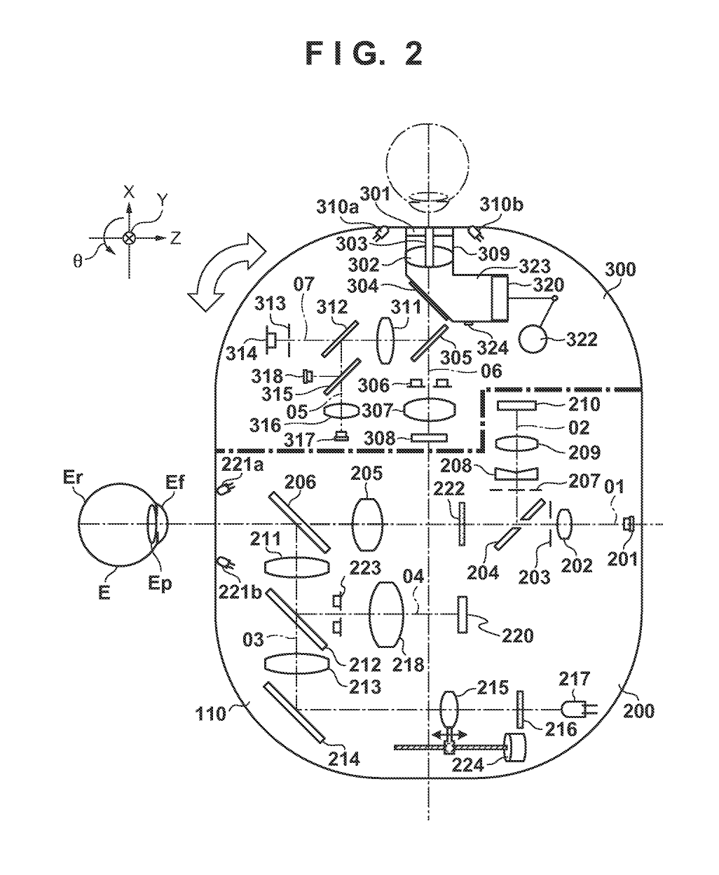

The optometric unit 110 includes an optical system for measurement, observation, and the like of an eye to be examined as an inspection target. FIG. 2 shows the arrangement of an optical system in the optometric unit 110 in the ophthalmic apparatus according to this embodiment. The optical system in the optometric unit 110 includes a first optical system 200 (first optometric unit) for inspecting the first eye characteristic and a second optical system 300 (second optometric unit) for inspecting the second eye characteristic different from the first eye characteristic of the eye to be examined.

The first optical system 200 is an optical system for inspecting the ocular refractive power of the eye to be examined. A projection lens 202, a stop 203 almost conjugate to a pupil Ep of the eye E, a perforated mirror 204, and a lens 205 are arranged on an optical path 01 extending from an ocular refractive power measurement light source 201 for emitting light with a wavelength of 880 nm to the eye E. In addition, a dichroic mirror 206 is disposed next to the above components on the optical path. The dichroic mirror 206 totally reflects infrared and visible light with wavelengths of 880 nm or more from the eye E side, and partly reflects a light beam with a wavelength of 880 nm or more.

A stop 207 which includes an annular slit and is almost conjugate to the pupil Ep, a light beam spectral prism 208, a lens 209, an image sensor 210 are sequentially disposed on an optical path 02 in the reflecting direction of the perforated mirror 204.

The above optical system is used for ocular refractive power measurement, in which the stop 203 restricts the light beam emitted from the ocular refractive power measurement light source 201. The projection lens 202 performs primary image formation in front of the lens 205. The resultant light beam is transmitted through the lens 205 and the dichroic mirror 206 and projected onto the pupil center of the eye E.

The reflected light of the projected light beam passes through the pupil center and enters the lens 205 again. The incident light beam is transmitted through the lens 205 and then reflected by the periphery of the perforated mirror 204.

The stop 207 almost conjugate to the pupil Ep of the eye to be examined and the light beam spectral prism 208 pupil-split the reflected light beam. The resultant light beam is projected as a ring image on the light-receiving surface of the image sensor 210. If the eye E is a normal-sighted eye, this ring image becomes a predetermined circle. If the eye E is a near-sighted eye, the projected image becomes a circle smaller than that originating from the normal-sighted eye. If the eye E is a far-sighted eye, the projected image becomes a circle larger than that originating from the normal-sighted eye. If the eye E has astigmatism, the ring image becomes an ellipse, with the angle defined by the horizontal axis and the ellipse representing an astigmatic axis angle. A refractive power is obtained based on this elliptic coefficient.

On the other hand, a visual fixation target projection optical system and an alignment light-receiving optical system used for both anterior ocular segment observation and alignment detection are arranged in the reflection direction of the dichroic mirror 206.

A lens 211, a dichroic mirror 212, a lens 213, a folding mirror 214, a lens 215, a visual fixation target 216, and a visual fixation target illumination light source 217 are sequentially arranged on an optical path 03 of the visual fixation target projection optical system.

At the time of visual fixation guidance, the projection light beam emitted from the visual fixation target illumination light source 217 in an ON state illuminates the visual fixation target 216 from the back side. The light beam is then projected onto a fundus Er of the eye E through the lens 215, the folding mirror 214, the lens 213, the dichroic mirror 212, and the lens 211.

Note that a visual fixation target guide motor 224 can move the lens 215 in the optical axis direction so as to implement a fogging state by performing visual fixation guidance for the eye E.

The alignment prism stop 223 which is inserted and removed by an alignment prism stop insertion/removal solenoid 411, an imaging lens 218, and an image sensor 220 are sequentially arranged on an optical path 04 in the reflecting direction of the dichroic mirror 212.

Inserting and removing the alignment prism stop 223 can perform alignment when the alignment prism stop 223 is located on the optical path 04 and can perform anterior ocular segment observation or transillumination observation when the alignment prism stop 223 is retracted from the optical path.

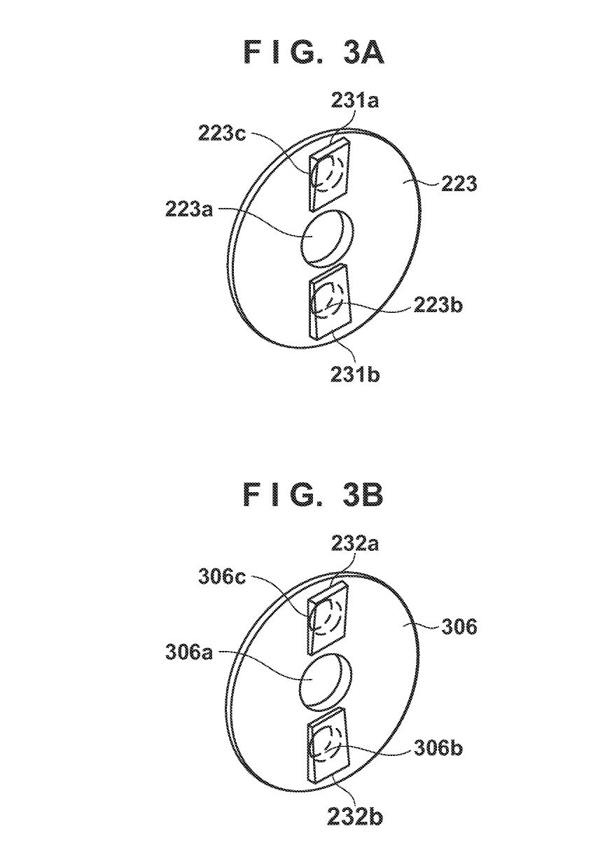

FIG. 3A shows the shape of the alignment prism stop 223. The disk-like stop plate is provided with three aperture portions 223a, 223b, and 223c. Alignment prisms 231a and 231b which transmit only light beams near a wavelength of 880 nm are bonded to the aperture portions 223a and 223b on the dichroic mirror 212 side.

Referring back to FIG. 2, anterior ocular segment illumination light sources 221a and 221b having a wavelength of about 780 nm are arranged diagonally in front of the anterior ocular segment of the eye E. The light beams of anterior ocular segment images of the eye E illuminated by the anterior ocular segment illumination light sources 221a and 221b are formed into images on the light-receiving sensor surface of the image sensor 220 via the dichroic mirror 206, the lens 211, the dichroic mirror 212, and the aperture portion 223a in the center of the alignment prism stop.

The light source used for alignment detection is also used as the ocular refractive power measurement light source 201. At the time of alignment, a diffuser panel insertion/removal solenoid 410 inserts a translucent diffuser panel 222 in an optical path.

The position at which the diffuser panel 222 is inserted is almost the primary imaging position of the projection lens 202 of the ocular refractive power measurement light source 201 and also coincides with the focal position of the lens 205. With this arrangement, an image of the ocular refractive power measurement light source 201 is temporarily formed on the diffuser panel 222. This image becomes a secondary light source and is projected from the lens 205 as a thick parallel light beam toward the eye E.

This parallel light beam is reflected by a cornea Ef of the eye to be examined and forms a bright spot image. The dichroic mirror 206 partly reflects this light beam again. This light beam is reflected by the dichroic mirror 212 through the lens 211, transmitted through the aperture portion 223a and alignment prisms 231a and 231b of the alignment prism stop, and focused by the imaging lens 218 to be formed into an image on the image sensor 220.

Light beams having a wavelength of 780 nm or more from the anterior ocular segment illumination light sources 221a and 221b pass through the aperture portion 223a in the center of the alignment prism stop 223. The anterior ocular segment image reflected light beams illuminated by the anterior ocular segment illumination light sources 221a and 221b propagate along the observation optical system like the path of a reflected light beam from the cornea Ef. These light beams are formed into images on the image sensor 220 by the imaging lens 218 through the aperture portion 223a of the alignment prism stop 223.

The light beam transmitted through the alignment prism 231a is refracted downward, and the light beam transmitted through the alignment prism 231b is refracted upward. It is possible to align the eye E in accordance with the positional relationship between these light beams passing through the stop.

While the alignment prism stop 223 and the cornea stop are retracted from the optical path 04, the dichroic mirror 206 reflects part of a light beam from the pupil area illuminated by the light beam emitted from the ocular refractive power measurement light source 201 and reflected by the fundus Er. This light beam is reflected by the dichroic mirror 212 through the lens 211. The imaging lens 218 then projects the light beam onto the image sensor 220. This light beam allows the observation of the eye E.

A second optical system 300 is an optical system for inspecting the eye pressure of an eye to be examined. On a light-receiving optical path and alignment detection optical path 06 of an observation optical system for the eye E, a nozzle 303 is disposed on the central axis of a plane parallel glass plate 301 and objective lens 302. An air chamber 323, an observation window 304, a dichroic mirror 305, a prism stop 306, an imaging lens 307, and an image sensor 308 are sequentially arranged behind the objective lens 302.

An objective lens barrel 309 supports the plane parallel glass plate 301 and the objective lens 302. Extraocular illumination light sources 310a and 310b for illuminating the eye E are arranged outside the objective lens barrel 309.

A relay lens 311, a half mirror 312, an aperture 313, and a light-receiving element 314 are arranged, in the reflecting direction of the dichroic mirror 305, on an optical path 07 of a deformation detection light-receiving optical system when the cornea Ef deforms in the visual axis direction. Note that the aperture 313 is disposed at a position at which it is conjugate to a cornea reflected image of an eye pressure measurement light source 317 (to be described later) at the time of predetermined deformation.

The relay lens 311 is designed to form a cornea reflected image almost equal in size to the aperture 313 when a cornea Ec deforms into a predetermined shape.

A half mirror 315 and a projection lens 316 are arranged, in the incident direction of the half mirror 312, on an optical path 05 of a measurement light source projection optical system for measuring the deformation of the cornea Ef. In addition, an eye pressure measurement light source 317 formed from a near-infrared LED used for both measurement and alignment for the eye E is disposed on the above optical path. Furthermore, a visual fixation light source 318 formed from an LED for visual fixation by an object is disposed in the incident direction of the half mirror 315.

A piston 320 is fitted in the objective lens barrel 309 forming part of the air chamber 323. A solenoid 322 drives the piston 320. Note that a pressure sensor 324 for monitoring an internal pressure is arranged in the air chamber 323.

(External Dimensions)

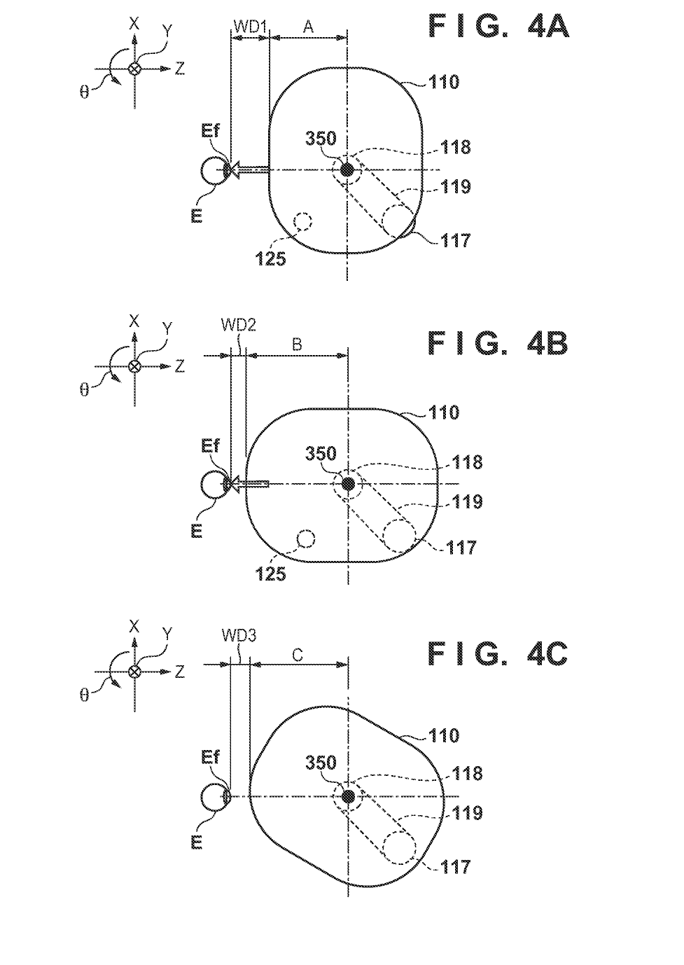

FIGS. 4A to 4C are plan views of the optometric unit 110. FIG. 4A shows the positional relationship between the optometric unit 110 and the eye E at the time of measurement of an ocular refractive power by the first optical system 200. FIG. 4B shows the positional relationship between the optometric unit 110 and the eye E at the time of measurement of an eye pressure by the second optical system 300. Let WD1 be an operating distance at the time of measurement of an ocular refractive power by the first optical system 200, that is, the distance from a cornea vertex Ef of the eye E to the first optical system output-side end portion of the optometric unit 110, and A be the distance from a rotation center 350 to the first optical system output-side end portion of the optometric unit 110. In addition, let WD2 be an operating distance at the time of measurement of an eye pressure by the second optical system 300, that is, the distance from the cornea vertex Ef of the eye E to the second optical system output-side end portion of the optometric unit 110, and B be the distance from the rotation center 350 to the second optical system output-side end portion of the optometric unit 110. In this case, the optometric unit 110 and the rotation center 350 are configured to satisfy WD1+A=WD2+B. FIG. 4C shows the positional relationship between the eye E and the optometric unit 110 during rotational movement. The external dimensions of the optometric unit 110 except for the first and second optical system output-side end portions are configured such that an external dimension C from the rotation center 350 keeps a distance WD3 at which the optometric unit 110 does not come into contact with any protruding portion of the object during rotational movement.

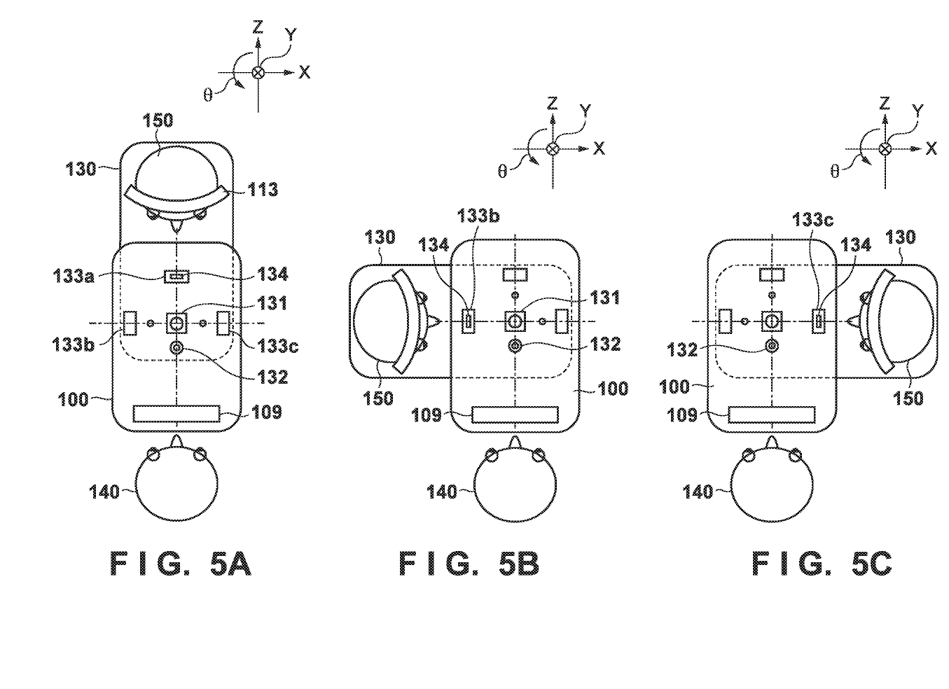

FIGS. 5A to 5C are plan views for explaining the moving mechanism of the face support unit 130. FIG. 5A shows the positional relationship between the base 100 and the face support unit 130 when an examiner 140 faces an object 150. At this time, the positioning stopper 132 positions the face support unit 130. While the positioning stopper 132 is positioning the face support unit 130, its movement is restricted. In addition, at this time, a dog 134 fixed on the face support unit has turned on a position detection sensor 133a.

FIG. 5B shows the positional relationship between the base 100 and the face support unit 130 when the object 150 is located on the left side of the examiner 140. When the positioning stopper 132 is removed in the state shown in FIG. 5A, the restriction of the movement of the face support unit 130 is canceled. The face support driving motor 131 (face support moving unit) drives the face support unit 130 to rotationally move relative to the base 100 from the state in FIG. 5A in the +Θ direction (counterclockwise direction). The positioning stopper then fixes the face support unit 130 at the position in FIG. 5B. At this time, the dog 134 fixed on the face support unit has turned on a position detection sensor 133b.

FIG. 5C shows the positional relationship between the base 100 and the face support unit 130 when the object 150 is located on the right side of the examiner 140. When the positioning stopper 132 is removed in the state shown in FIG. 5A, the restriction of the movement of the face support unit 130 is canceled. The face support driving motor 131 (face support moving unit) drives the face support unit 130 to rotationally move relative to the base 100 from the state in FIG. 5A in the −Θ direction (clockwise direction). The positioning stopper then fixes the face support unit 130 at the position in FIG. 5C. At this time, the dog 134 fixed on the face support unit has turned on a position detection sensor 133c. When rotating the face support unit 130, it is possible that the rotation center position of the face support unit 130 almost coincides with the rotation center 350 when the optometric unit 110 is located at the origin position (the center position of each axis). This makes it possible to keep the distance from the rotation center 350 to the eye E, even when the optometric unit 110 rotates, and reduce an unnecessary moving amount at the time of measurement. This arrangement allows the examiner 140 to move the face support unit 130, before actual measurement, to a position which facilitates the operation. For example, the examiner can change the position of the face support unit to the position in FIG. 5B or 5C to allow him/her to perform eyelid retraction on the dominant hand side. In addition, disposing the face support unit 130 at the position in FIG. 5B or 5C allows the examiner to perform measurement even if the face support unit is disposed against a wall or at a corner portion in the installation area of the apparatus. This increases the degree of freedom of installation layout.

(System Block Diagram)

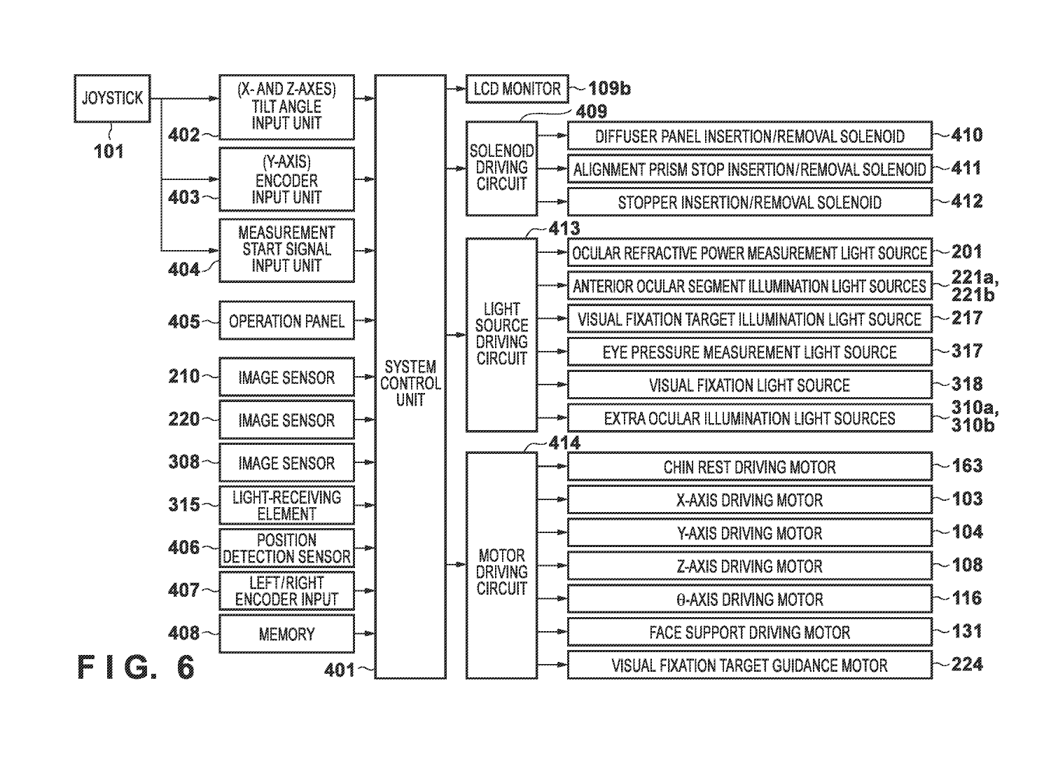

FIG. 6 is a system block diagram of the ophthalmic apparatus. A system control unit 401 controls the overall system. The system control unit 401 includes a program storage unit and a data storage unit storing data for correcting eye pressure values, ocular refractive power values, and the like. The system control unit 401 also includes an input/output control unit which controls input/output operation with various types of devices and an arithmetic processing unit which computes the data obtained from various types of devices.

A tilt angle input unit 402, an encoder input unit 403, and a measurement start signal input unit 404 are connected to the system control unit 401. The system control unit 401 receives instructions (signals) from the joystick 101 for positioning the optometric unit 110 to the eye E and starting measurement via the tilt angle input unit 402, the encoder input unit 403, and the measurement start signal input unit 404. The tilt angle input unit 402 detects tilt angles when the examiner tilts the joystick 101 back and forth and left and right and inputs detected tilt angles to the system control unit 401. The encoder input unit 403 accepts encoder signals from various types of driving motors when the examiner operates the joystick 101 to rotate the respective types of driving motors, and inputs the signals to the system control unit 401. The measurement start signal input unit 404 accepts a signal transmitted when the examiner presses the measurement start button of the joystick 101, and inputs the signal to the system control unit 401.

In addition, a print button, a chin rest up/down button, and the like are arranged on an operation panel 405 on the base 100. When the examiner perform button input operation, the panel notifies the system control unit 401 of a corresponding signal. Furthermore, signals from the respective types of position detection sensors 406 including the position detection sensors 133a, 133b, and 133c (detection units) are notified to the system control unit 401 when the sensors are turned on.

A memory 408 stores the anterior ocular segment image of the eye E captured by the image sensor 220. The system control unit 401 extracts the pupil and cornea reflected images of the eye E from the image stored in the memory 408 and performs alignment detection. In addition, the anterior ocular segment image of the eye E captured by the image sensor 220 is combined with characters and graphic data to display the anterior ocular segment image and measurement values on the LCD monitor (display unit 109b).

The memory 408 stores the ring image for ocular refractive power calculation captured by the image sensor 210.

The system control unit 401 issues commands via a solenoid driving circuit 409 to control the driving of the solenoids 410 to 412.

In addition, the X-axis driving motor 103, the Y-axis driving motor 104, the Z-axis driving motor 108, the chin rest driving motor 163, the Θ-axis driving motor 116, the face support driving motor 131, and the visual fixation target guidance motor 224 are connected to a motor driving circuit 414. The motor driving circuit 414 accepts commands from the system control unit 401 and drives the respective types of motors.

The ocular refractive power measurement light source 201, the anterior ocular segment illumination light sources 221a and 221b for ocular refractive power measurement, the visual fixation target illumination light source 217, the eye pressure measurement light source 317, the visual fixation light source 318, and the extraocular illumination light sources 310a and 310b for eye pressure measurement are connected to a light source driving circuit 413. The light source driving circuit 413 accepts commands from the system control unit 401 and controls ON/OFF operation and light amount changing operation of the respective types of light sources.

The operation of the apparatus having the above arrangement will be described.

(Ocular Refractive Power Measurement)

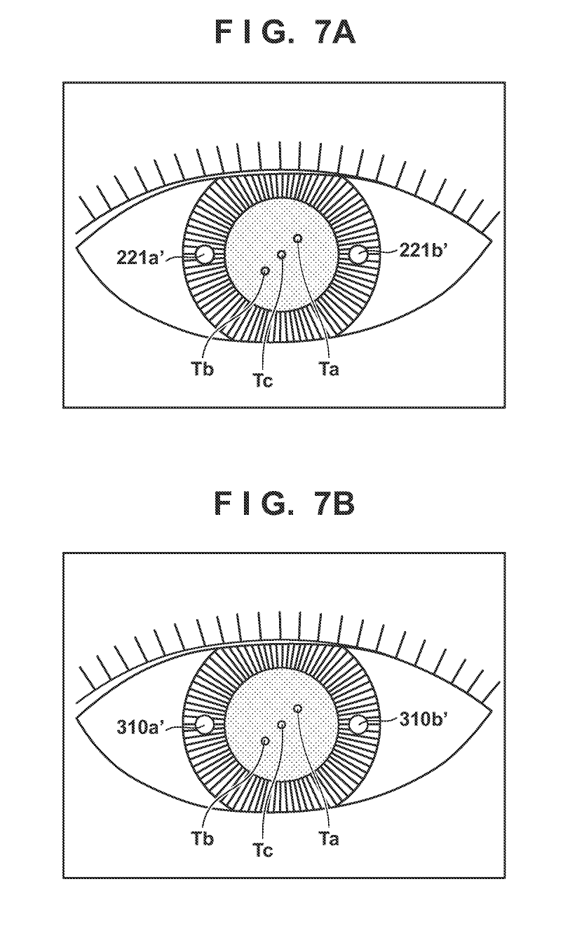

As shown in FIG. 7A, at the time of alignment, the aperture portions 223a, 223b, and 223c of the alignment prism stop 223 and the alignment prisms 231a and 231b split the cornea bright spot formed by the cornea Ef. The image sensor 220 captures, as index images Ta, Tb, and Tc, the cornea bright spots, the eye E illuminated by the anterior ocular segment illumination light sources 221a and 221b, and bright spot images 221a′ and 221b′ of the anterior ocular segment illumination light sources 221a and 221b.

Alignment is executed in two steps, namely rough alignment of performing rough positioning and fine alignment of performing fine positioning.

Rough alignment uses the eye E and the bright spot images 221a′ and 221b′ of the anterior ocular segment illumination light sources 221a and 221b. Upon detecting the eye E and the bright spot images 221a′ and 221b′, the system control unit 401 controls the motor driving circuit 414. The system control unit 401 then drives the optometric unit 110 up and down and left and right so as to align the bright spot images 221a′ and 221b′ with the pupil center of the eye E in the X and Y directions.

The system control unit 401 then calculates Z-coordinates and areas of the bright spot images 221a′ and 221b′ and drives the optometric unit 110 in the back-and-forth direction so as to align the images with a predetermined position, thereby performing rough positioning.

Fine alignment uses the index images Ta, Tb, and Tc. Upon detecting the three bright spots Ta, Tb, and Tc, the system control unit 401 controls the motor driving circuit 414. The system control unit 401 drives the optometric unit 110 up and down and left and right so as to align the middle bright spot Tc with the center of the eye E. The system control unit 401 drives the optometric unit 110 back and forth so as to align the bright spots Ta and Tb with the bright spot Tc in the vertical direction, and completes the alignment upon aligning the three cornea bright spots Ta, Tb, and Tc with each other in the vertical direction.

To measure an ocular refractive power, the system control unit 401 retracts the diffuser panel 222, which has been inserted in the optical path 01 for automatic alignment, from the optical path 01. The system control unit 401 adjusts the light amount of the ocular refractive power measurement light source 201 and projects a measurement light beam on the fundus Er of the eye E.

The image sensor 210 receives reflected light from the fundus along the optical path 02. The stop 207 having a ring-like aperture projects the captured fundus image into a ring image owing to the refractive power of the eye to be examined. The memory 408 stores this ring image.

The system control unit 401 calculates the barycentric coordinates of the ring image stored in the memory 408 and obtains an ellipse equation by a known method. The system control unit 401 calculates the major and minor axes and major-axis gradient of the obtained ellipse and calculates the ocular refractive power of the eye E.

Note that ocular refractive powers corresponding to the major and minor axes of the obtained ellipse and the relationship between the angle of the ellipse axis and the astigmatic axis on the light-receiving surface of the image sensor 210 have been corrected in advance in the manufacturing process of the apparatus.

The system control unit 401 drives the visual fixation target guidance motor 224 via the motor driving circuit 414 to move the lens 215 to a position corresponding to a refractive power corresponding to the obtained ocular refractive power, and presents the eye E with the visual fixation target 216 with a degree of refraction corresponding to the degree of refraction of the eye E.

Subsequently, the system control unit 401 moves the lens 215 to a predetermined distance, fogs the visual fixation target 216, and turns on the measurement light source again to measure a refractive power. It is possible to obtain the final measurement value, at which the refractive power becomes stable, by repeating measurement of a refractive power, fogging of the visual fixation target 216, and measurement of a refractive power in this manner.

(Eye Pressure Measurement)

As shown in FIG. 7B, at the time of alignment for eye pressure measurement, aperture portions 306a, 306b, and 306c of the prism stop 306 and prisms 232a and 232b shown in FIG. 3B split the cornea bright spot formed by the cornea Ef. The image sensor 308 captures, as index images Ta, Tb, and Tc, the cornea bright spots, the eye E illuminated by the extraocular illumination light sources 310a and 310b, the cornea bright spots, the eye E illuminated by the extraocular illumination light sources 310a and 310b, and bright spot images 310a′ and 310b′ of the extraocular illumination light sources 310a and 310b. The following operation is the same as that performed at the time of alignment for ocular refractive power measurement.

The system control unit 401 performs eye pressure measurement after the completion of alignment. The system control unit 401 drives the solenoid 322. The piston 320 raised by the solenoid 322 compresses the air in the air chamber 323 to jet an air pulse from the nozzle 303 to the cornea Ef of the eye E.

The pressure signal detected by the pressure sensor 324 of the air chamber 323 and the light reception signal from the light-receiving element 314 are output to the system control unit 401. The system control unit 401 then calculates an eye pressure value from the peak value of the light reception signal and the pressure signal at this time.

(Explanation of Operation in Automatic Driving)

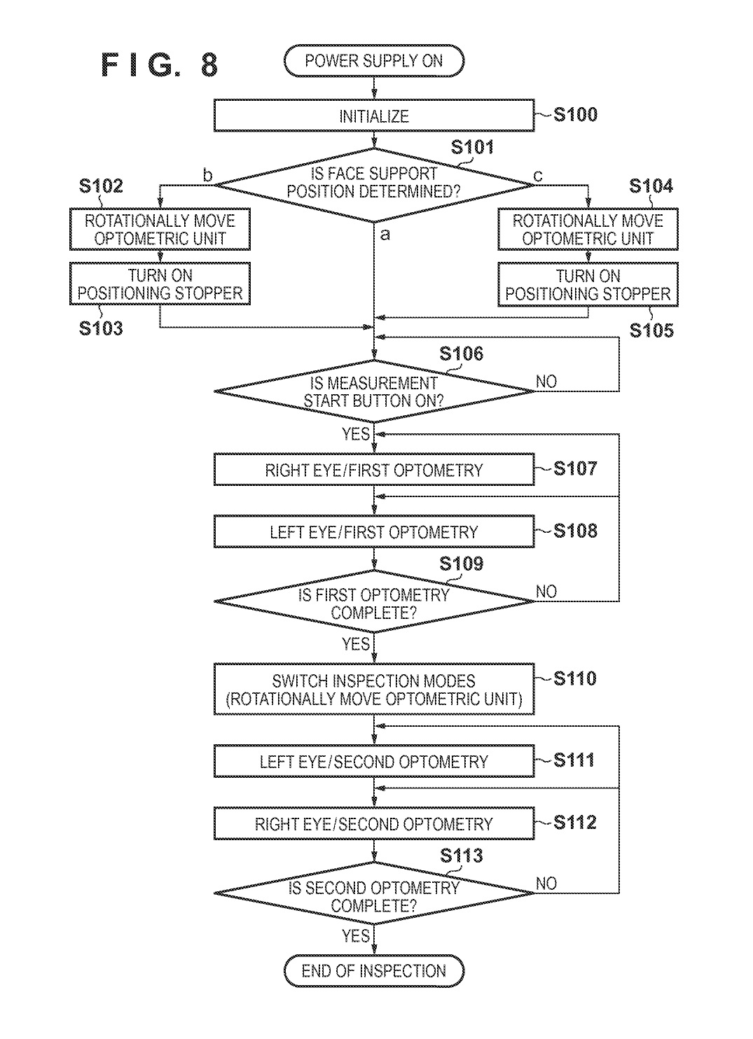

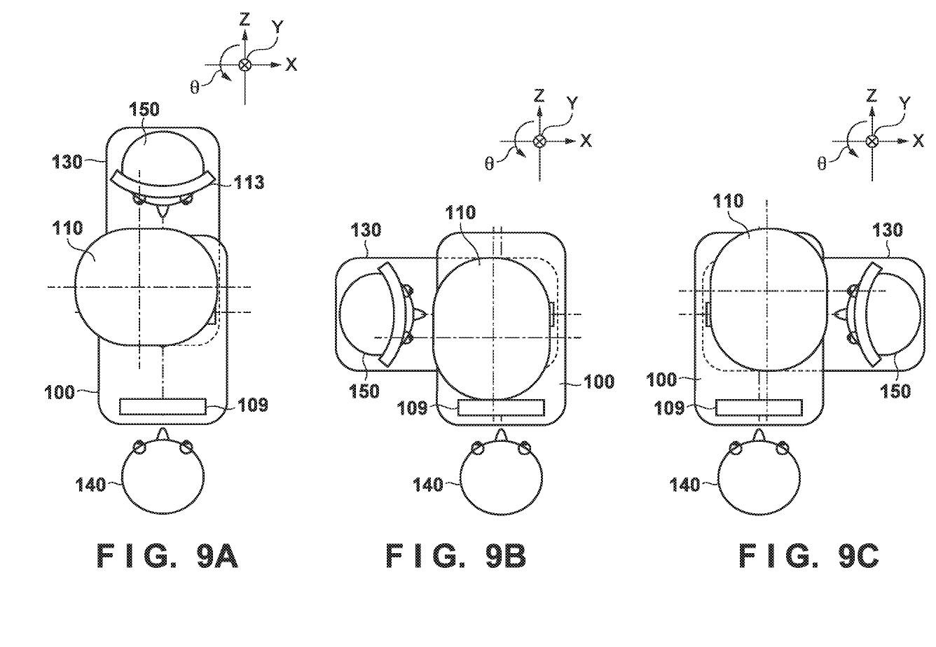

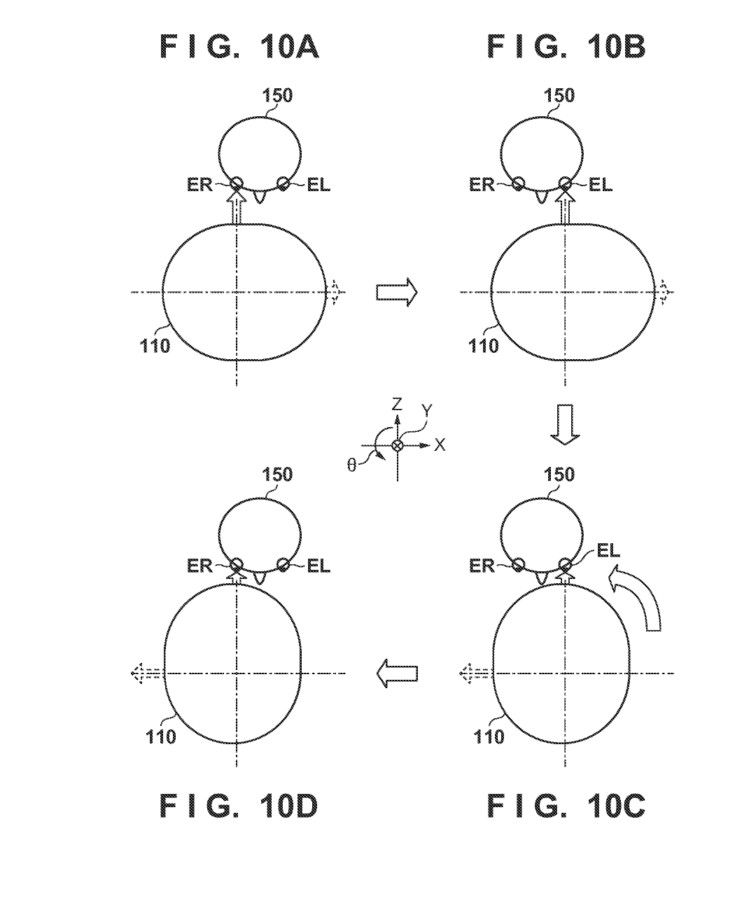

Operation in automatic driving as operation at the time of optometry in the ophthalmic apparatus having the above arrangement will be described with reference to the flowchart of FIG. 8 and the operation charts of FIGS. 9A to 9C and 10A to 10D.

When the examiner 140 turns on the power supply to start up the ophthalmic apparatus, the ophthalmic apparatus initializes the respective types of devices (step S100). Thereafter, the system control unit 401 (determination unit) determines the position of the face support unit 130 (step S101).

If the position detection sensor 133a (detection unit) is ON (step S101-a), the optometric unit 110 does not rotationally move and moves to the position where it measures the ocular refractive power of a right eye ER to be examined of the object 150 in FIG. 9A, thereby completing preparation. If the position detection sensor 133b (detection unit) is ON (step S101-b), the Θ-axis driving motor 116 rotationally moves the optometric unit 110 (step S102). After the positioning stopper 125 fixes the optometric unit 110 (step S103), the optometric unit 110 moves to the position where it measures the ocular refractive power of the right eye ER of the object 150 in FIG. 9B, thereby completing preparation. If the position detection sensor 133c (detection unit) is ON (step S101-c), the Θ-axis driving motor 116 rotationally moves the optometric unit 110 (step S104). After the positioning stopper 125 fixes the optometric unit 110 (step S105), the optometric unit 110 moves to the position where it measures the ocular refractive power of the right eye ER of the object 150 in FIG. 9C, thereby completing preparation.

In this state, the examiner 140 makes the object 150 rest his/her chin on the chin rest 112 and press his/her forehead against the forehead rest portion of the face support frame 113 to fix the eye E. The examiner 140 then selects the full automatic mode by operating a switch (not shown) on the LCD monitor (display unit 109b). The examiner 140 sets the pupil center of the right eye ER in the observation range of the LCD monitor (display unit 109b) by tiling the joystick 101, as needed. When the examiner presses the measurement start button 121 in this state (YES in step S106), the apparatus starts automatic measurement (step S106). If the examiner does not press the measurement start button 121 (NO in step S106), the apparatus enters a standby state to press the measurement start button 121.

When the examiner presses the measurement start button (YES in step S106), the apparatus starts rough alignment to perform rough positioning for ocular refractive power measurement. Upon completing the rough alignment, the apparatus starts fine alignment to perform more precise positioning. Upon completing the fine alignment, the apparatus measures the ocular refractive power of the right eye ER of the object a predetermined number of times (step S107 and FIG. 10A: right eye/first optometry). Upon measuring the ocular refractive power of the right eye to be examined, the apparatus moves the optometric unit 110 in the X and Z directions by necessary amounts, and measures the ocular refractive power of a left eye EL to be examined of the object a predetermined number of times (step S108 and FIG. 10B: left eye/first optometry). The apparatus repeats measurement in steps S107 and S108 until performing ocular refractive power measurement a predetermined number of times (step S107, step S108, and NO in step S109). Upon completely measuring the ocular refractive powers of the left and right eyes a predetermined number of times (YES in step S109), the apparatus moves the optometric unit 110 in the Θ direction to switch optometry from ocular refractive power measurement from eye pressure measurement (step S110). At this time, the optometric unit 110 rotationally moves so as not to come into contact with any protruding portion (for example, the nose) of the object. More specifically, the output-side end portion of the eye pressure measurement optical system of the optometric unit 110 rotationally moves from the left ear side to the nose side of the object (FIG. 10C). This makes it possible to perform quick switching operation with only the Θ axis while preventing interference with any protruding portion of the object. In addition, since switching is performed by only rotating operation with only the Θ axis and the distance from the rotation center to the eye E is constant, the positions of the optometric unit 110 in the X and Y directions are reproduced and an operating distance in the Z direction required for eye pressure measurement can be automatically obtained. This makes it unnecessary to perform rough alignment after switching operation, and can further shorten the optometry time. After switching operation, the apparatus starts fine alignment for the left eye EL of the object. Upon completing the fine alignment, the apparatus measures the eye pressure of the left eye EL a predetermined number of times (step S111 and FIG. 100: left eye/second optometry). At this time, to perform more accurate measurement, the examiner 140 often performs eyelid retraction to make the eyelid of the left eye EL of the object 150 open. In the case of the arrangement of FIG. 9B, the examiner 140 performs eyelid retraction with his/her right hand while checking the left eye EL of the object 150 by the naked eye. In the case of the arrangement of FIG. 9C, the examiner 140 can perform eyelid retraction with his/her right hand while checking the left eye EL of the object 150 by the naked eye. This allows the examiner 140 to reliably and comfortably perform eyelid retraction as compared with operation in the arrangement shown in FIG. 9A. Upon completing eye pressure measurement of the left eye EL, the apparatus moves the optometric unit 110 in the X and Z directions by necessary amounts and measures the eye pressure of the right eye ER a predetermined number of times (step S112 and FIG. 10D: right eye/second optometry). At this time, the examiner 140 performs eyelid retraction for the right eye ER. The apparatus repeats measurement in steps S111 and S112 until performing eye pressure measurement a predetermined number of times (step S111, step S112, and NO in step S113). Upon completing eye pressure measurement of the left and right eyes a predetermined number of times (YES in step S113), the apparatus terminates the inspection.

### Second Embodiment

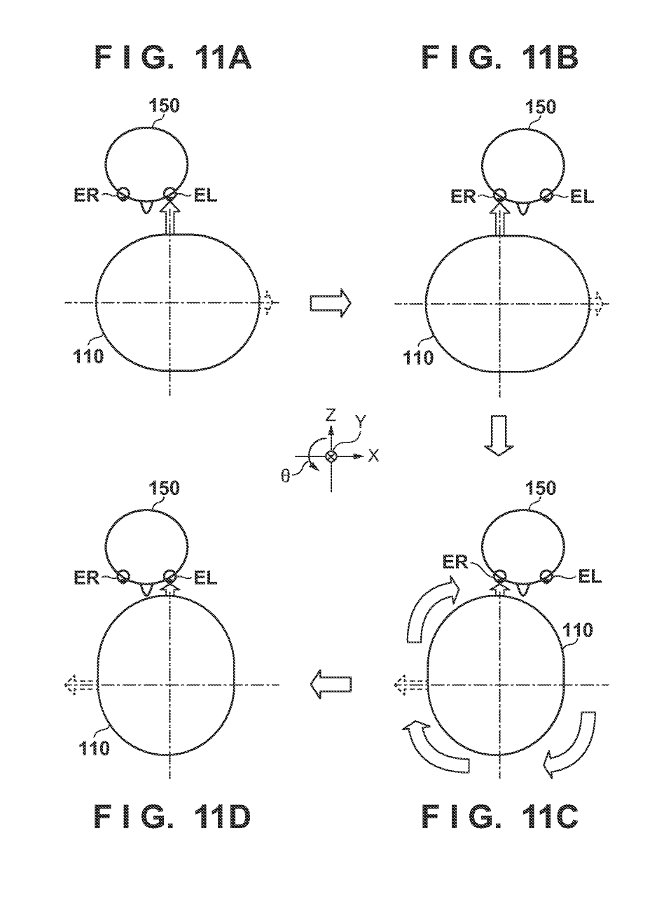

FIGS. 11A to 11D are views for explaining the second embodiment in a case in which ocular refractive power measurement starts from the left eye to be examined. In this case, upon performing ocular refractive power measurement in the order of a left eye EL to be examined and a right eye ER to be examined (FIGS. 11A and 11B), the apparatus moves an optometric unit 110 in the Θ direction to switch optometry from ocular refractive power measurement to eye pressure measurement. At the time of this switching operation, a system control unit 401 rotates the optometric unit 110 to move it from the output-side end portion of the eye pressure measurement optical system to the right ear side so as not to come into contact with any protruding portion (for example, the nose) of the object. This makes it possible to quickly perform switching operation with only the Θ axis while preventing interference with any protruding portion of the object. In addition, since switching is performed by only rotating operation with only the Θ axis and the distance from the rotation center to the eye E is constant, the positions of the optometric unit 110 in the X and Y directions are reproduced and an operating distance in the Z direction required for eye pressure measurement can be automatically obtained. This makes it unnecessary to perform rough alignment after switching operation, and can further shorten the optometry time. After switching operation, the apparatus starts fine alignment for the right eye ER of the object. Upon completing fine alignment, the apparatus measures the eye pressure of the right eye ER a predetermined number of times (FIG. 11C). Upon completing eye pressure measurement of the right eye ER, the apparatus moves the optometric unit 110 in the X and Z directions by necessary amounts and measures the eye pressure of the left eye EL a predetermined number of times (FIG. 11D).

The ophthalmic apparatus according to the embodiment of the present invention is a composite type ophthalmic apparatus. When switching one type of inspection by the optometric unit 110 to a different type of inspection, the driving mechanism in the O-axis direction (optometric unit moving unit) moves the optometric unit 110 in a rotational direction relative to a base 100 (apparatus fixing unit). For the sake of simplicity, according to the above embodiments, the functions to be combined are limited to the ocular refractive power function and the eye pressure function. However, the present invention can be applied to an ophthalmic apparatus which additionally includes other optometry functions such as a cornea curvature radius measurement function and a cornea thickness measurement function. In addition, optometry functions to be added are not limited to measurement functions. The present invention can be applied to general ophthalmic apparatuses which perform inspections concerning the eye to be examined, for example, a fundus camera and an OCT (Optical Coherent Tomography) apparatus.

Although the rotating mechanism for the optometric unit 110 in this embodiment is a mechanism using pulleys and belts, the scope of the present invention is not limited to this arrangement. For example, the output shaft of a motor may be directly coupled to the optometric unit and rotate. Alternatively, a rotating mechanism may be formed by using other mechanisms such as a chain driving mechanism.

In this embodiment, the motor is used for the moving mechanism of the face support unit to allow automatic movement of the mechanism. However, the present invention is not limited to this, and the face support unit 130 may be manually moved. In addition, this embodiment uses the automatic driving mechanism as the positioning stopper. However, the scope of the present invention is not limited to this arrangement. For example, it is possible to fix the face support unit by using a mechanism such as a ball plunger mechanism. In addition, with regard to the detection of the position of the face support unit 130, this embodiment has exemplified the case in which the face support unit 130 can move to three positions by using microsensors as position detection sensors. However, the scope of the present invention is not limited to this arrangement. For example, it is possible to dispose the face support unit 130 at an arbitrary position by allowing to detect an arbitrary position by using an encoder or the like. This can increase the degree of freedom of installation layout.

The order of inspection is not limited to ocular refractive power measurement→eye pressure measurement and right eye optometry→left eye optometry. The present invention can be applied to arbitrary orders of inspection. The driving mode to be used is not limited to full automatic driving. The present invention can be applied to the manual driving mode, semi-automatic driving mode, and the like.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-093398, filed Apr. 16, 2012, which is hereby incorporated by reference herein in its entirety.

What is claimed is:

1. An ophthalmic apparatus including an apparatus fixing unit, an optometric unit configured to move relative to said apparatus fixing unit, and a face support unit configured to fix an eye to be examined as an inspection target of said optometric unit, the apparatus comprising:
a face support moving unit configured to move said face support unit relative to said apparatus fixing unit; and
an optometric unit moving unit configured to move said optometric unit relative to said apparatus fixing unit.

2. The apparatus according to claim 1, wherein said optometric unit moving unit rotationally moves said optometric unit around a rotation axis relative to said apparatus fixing unit.

3. The apparatus according to claim 2, wherein said face support moving unit rotationally moves said face support unit around a rotation axis relative to said apparatus fixing unit, and
the rotation axis around which rotational movement performed by said optometric unit moving unit coincides with the rotation axis around which rotational movement is performed by said face support moving unit.

4. The apparatus according to claim 1, further comprising:
a detection unit configured to detect a position of said face support unit; and
a determination unit configured to determine a position of said face support unit based on detection by said detection unit,
wherein said optometric unit moving unit moves said optometric unit in accordance with a position determined by said determination unit, and
said optometric unit inspects the eye to be examined after said optometric unit moving unit completes movement.

5. The apparatus according to claim 1, further comprising a display unit configured to display an anterior ocular segment image of the eye to be examined which is inspected by said optometric unit,
wherein said display unit is provided on said apparatus fixing unit.

6. The apparatus according to claim 1, wherein the ophthalmic apparatus comprises a composite type ophthalmic apparatus, and
said optometric unit comprises
a first optometric unit configured to include an optical system for inspecting a first eye characteristic of the eye to be examined, and
a second optometric unit configured to include an optical system for inspecting a second eye characteristic different from the first eye characteristic of the eye to be examined.

7. The apparatus according to claim 6, wherein said first optometric unit of said optometric unit comprises an optometric unit including an optical system configured to inspect an ocular refractive power of the eye to be examined, and said second optometric unit of said optometric unit comprises an optometric unit including an optical system configured to inspect an eye pressure of the eye to be examined.
